(19) United States
(12) Patent Application Publication
(10) Pub. No.: US 2020/0308141 A1
(43) Pub. Date: Oct. 1, 2020

(54) SOLID STATE FORMS OF NERATINIB AND SALTS THEREOF

(71) Applicant: PLIVA HRVATSKA D.O.O., Zagreb (HR)

(72) Inventors: Igor AVDEJEV, Zagreb (HR); Edi TOPIC, Malinska (HR); Dijana Skalec SAMEC, Jastrebarsko (HR); Lidija LERMAN, Zagreb (HR); Vitomir VUSAK, Zagreb (HR)

(21) Appl. No.: 16/313,502

(22) PCT Filed: Jun. 27, 2017
(86) PCT No.: PCT/US2017/039392

§ 371 Date: Dec. 27, 2018

Related U.S. Application Data
(60) Provisional application No. 62/354,915, filed on Jun. 27, 2016.
(60) Provisional application No. 62/421,688, filed on Nov. 14, 2016.

(51) Int. Cl. C07D 401/12

(52) U.S. Cl. CPC C07D 401/12; C07B 2200/13

(57) ABSTRACT

Solid state forms of Neratinib and salts thereof, processes for preparation thereof and pharmaceutical compositions thereof are disclosed.

### FIELD OF THE INVENTION

The present disclosure relates to solid state forms of Neratinib and salts thereof, processes for preparation thereof and pharmaceutical compositions thereof.

### BACKGROUND OF THE INVENTION

Neratinib (or Neratinib base) has the chemical name (E)-N-{4-[3-Chloro-4-(2-pyridinylmethoxy)anilino]-3-cyano-7-ethoxy-6-quinolinyl}-4-(dimethylamino) -2-butenamide. Neratinib has the following chemical structure:

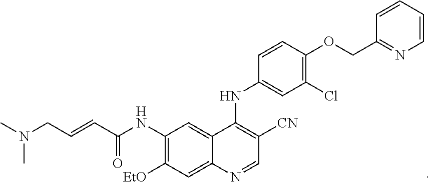 [SMILES: CCOc1cc2ncc(C#N)c(Nc3ccc(OCc4ccccn4)c(Cl)c3)c2cc1NC(=O)/C=C/CN(C)C]

Neratinib is apparently an irreversible inhibitor of the HER-2 receptor tyrosine kinase with potential antineoplastic activity. Neratinib is under investigation for the treatment of breast cancer and other solid tumors.

Neratinib is known from U.S. Pat. No. 7,399,865.

US20060270668 describes a formation of Neratinib salt, such as a fumarate or mesylate salt. According to US20060270668, this is in order to stabilize the molecule and render the compound more soluble as compared to the base. It is further described that the most preferred salt was a maleate salt.

Crystalline forms of Neratinib maleate (an anhydrous form, a monohydrate form, and a mixture of the anhydrous and the monohydrate forms (referred to as a partial hydrate form) are described in WO2009052264. A crystalline form of Neratinib maleate is described in WO2016110270. CN106831710A describes amorphous neratinib, amorphous neratinib maleate, and amorphous solid dispersions.

Polymorphism, the occurrence of different crystal forms, is a property of some molecules and molecular complexes. A single compound, like Neratinib, may give rise to a variety of polymorphs having distinct crystal structures and physical properties like melting point, thermal behaviors (e.g. measured by thermogravimetric analysis—“TGA”, or differential scanning calorimetry—“DSC”), powder X-ray diffraction (PXRD) pattern, infrared absorption fingerprint, Raman absorption fingerprint, and solid state (¹³C—) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, improving the dissolution profile, or improving stability (polymorph as well as chemical stability) and shelf-life. These variations in the properties of different salts and solid state forms may also provide improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to use variations in the properties and characteristics of a solid active pharmaceutical ingredient for providing an improved product.

Discovering new salts, solid state forms and solvates of a pharmaceutical product can provide materials having desirable processing properties, such as ease of handling, ease of processing, storage stability, and ease of purification or as desirable intermediate crystal forms that facilitate conversion to other salts or polymorphic forms. New salts, polymorphic forms and solvates of a pharmaceutically useful compound can also provide an opportunity to improve the performance characteristics of a pharmaceutical product (dissolution profile, bioavailability, etc.). It enlarges the repertoire of materials that a formulation scientist has available for formulation optimization, for example by providing a product with different properties, e.g., a different crystal habit, higher crystallinity or polymorphic stability which may offer better processing or handling characteristics, improved dissolution profile, or improved shelf-life.

For at least these reasons, there is a need for additional solid state forms (including solvated forms) of Neratinib and salts thereof.

### SUMMARY OF THE INVENTION

The present disclosure relates to solid state forms of Neratinib and salts thereof, to processes for preparation thereof, and to pharmaceutical compositions comprising these solid state forms.

The present disclosure also provides uses of the solid state forms of Neratinib and salts thereof for preparing other solid state forms of Neratinib, Neratinib salts and solid state forms thereof.

In another embodiment, the present disclosure encompasses the above described solid state forms of Neratinib and salts thereof for use in the preparation of pharmaceutical compositions and/or formulations, preferably for the treatment of HER2-positive solid tumors, including breast cancer and non-small cell lung cancer (NSCLC).

In another embodiment the present disclosure encompasses the use of the above described solid state forms of Neratinib and salts thereof for the preparation of pharmaceutical compositions and/or formulations.

The present disclosure further provides pharmaceutical compositions comprising the solid state forms of Neratinib and salts thereof according to the present disclosure.

In yet another embodiment, the present disclosure encompasses pharmaceutical formulations comprising the above described solid state forms of Neratinib and salts thereof and at least one pharmaceutically acceptable excipient.

The present disclosure encompasses processes to prepare said pharmaceutical formulations of Neratinib comprising combining the above solid state forms and at least one pharmaceutically acceptable excipient.

The solid state forms as defined herein, as well as the pharmaceutical compositions or formulations of the solid state form of Neratinib and salts thereof, can be used as medicaments, particularly for the treatment of HER2-positive solid tumors, including breast cancer and non-small cell lung cancer (NSCLC).

The present disclosure also provides methods of treating HER2-positive solid tumors, including breast cancer and non-small cell lung cancer (NSCLC); comprising administering a therapeutically effective amount of the solid state forms of Neratinib and salts thereof of the present disclosure, or at least one of the above pharmaceutical compositions or formulations, to a subject suffering from cancer, or otherwise in need of the treatment.

The present disclosure also provides uses of the solid state forms of Neratinib and salts thereof of the present disclosure, or at least one of the above pharmaceutical compositions or formulations for the manufacture of a medicament for treating HER2-positive solid tumors, including breast cancer and non-small cell lung cancer (NSCLC).

### BRIEF DESCRIPTION OF THE FIGURES

FIG. 1 shows a powder X-ray diffraction pattern (“powder XRD” or “PXRD”) of Neratinib base form B1 obtained in Example 2.

FIG. 2 shows a powder X-ray diffraction pattern of Neratinib base form B2 obtained in Example 4.

FIG. 3 shows a powder X-ray diffraction pattern of Neratinib base form B3 obtained in Example 1.

FIG. 4 shows a powder X-ray diffraction pattern of Neratinib base form B4 obtained in Example 6.

FIG. 5 shows a powder X-ray diffraction pattern of Neratinib maleate form T2 obtained in Example 8.

FIG. 6 shows a powder X-ray diffraction pattern of Neratinib base form B6 obtained in Example 9.

### DETAILED DESCRIPTION OF THE INVENTION

The present disclosure relates to solid state forms of Neratinib and salts thereof, processes for preparation thereof and pharmaceutical compositions comprising said solid state forms. The disclosure also relates to the conversion of Neratinib base or Neratinib salt and their solid state forms to other solid state forms of Neratinib base, Neratinib salts (for example Neratinib maleate) and solid state forms thereof.

The Neratinib base, Neratinib maleate and solid state forms thereof according to the present disclosure may have advantageous properties selected from at least one of: chemical or polymorphic purity, flowability, solubility, dissolution rate, bioavailability, morphology or crystal habit, stability—such as chemical stability as well as thermal and mechanical stability with respect to polymorphic conversion, stability towards dehydration and/or storage stability, a lower degree of hygroscopicity, low content of residual solvents and advantageous processing and handling characteristics such as compressibility, or bulk density.

A crystal form may be referred to herein as being characterized by graphical data “as depicted in” a Figure. Such data include, for example, powder X-ray diffractograms and solid state NMR spectra. As is well-known in the art, the graphical data potentially provides additional technical information to further define the respective solid state form (a so-called “fingerprint”) which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Neratinib and salts thereof referred to herein as being characterized by graphical data “as depicted in” a Figure will thus be understood to include any crystal forms of the Neratinib and salts thereof, characterized with the graphical data having such small variations, as are well known to the skilled person, in comparison with the Figure.

A solid state form (or polymorph) may be referred to herein as polymorphically pure or substantially free of any other solid state (or polymorphic) forms. As used herein in this context, the expression “substantially free of any other forms” will be understood to mean that the solid state form contains about 20% or less, about 10% or less, about 5% or less, about 2% or less, about 1% or less, about 0.5% or less, or about 0% of any other forms of the subject compound as measured, for example, by PXRD. Thus, solid state of Neratinib and Neratinib salts, preferably maleate salt, described herein as substantially free of any other solid state forms would be understood to contain greater than about 80% (w/w), greater than about 90% (w/w), greater than about 95% (w/w), greater than about 98% (w/w), greater than about 99% (w/w), greater than about 99.5% (w/w), or greater than about 0% (w/w) of the subject solid state form of Neratinib and Neratinib salts. Accordingly, in some embodiments of the disclosure, the described solid state forms of Neratinib and Neratinib salts may contain from about 1% to about 20% (w/w), or about 0.5% to about 20% (w/w), from about 5% to about 20% (w/w), or from about 5% to about 10% (w/w) of one or more other solid state forms of the same Neratinib and Neratinib salts.

The modifier “about” should be considered as disclosing the range defined by the absolute values of the two endpoints. For example, the expression “from about 2 to about 4” also discloses the range “from 2 to 4.” When used to modify a single number, the term “about” may refer to plus or minus 10% of the indicated number and includes the indicated number. For example, “about 10%” may indicate a range of 9% to 11%, and “about 1” means from 0.9-1.1.

As used herein, unless stated otherwise, PXRD peaks reported herein are preferably measured using CuK_{α} radiation, λ=1.5418 Å. Preferably, PXRD peaks reported herein are measured using CuK_{α} radiation, λ=1.54184 Å, at a temperature of 25±3° C.

As used herein, the term “isolated” in reference to solid state forms of Neratinib and Neratinib salts, preferably maleate salt, of the present disclosure corresponds to solid state forms of Neratinib and Neratinib salts that are physically separated from the reaction mixture in which it is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C. A process or step may be referred to herein as being carried out “overnight.” This refers to a time interval, e.g., for the process or step, that spans the time during the night, when that process or step may not be actively observed. This time interval is from about 8 to about 20 hours, or about 10 to about 18 hours, typically about 16 hours.

As used herein, the expression “wet crystalline form” refers to a polymorph that was not dried using any conventional techniques to remove residual solvent. Examples for such conventional techniques can be, but not limited to, evaporation, vacuum drying, oven drying, drying under nitrogen flow, etc.

As used herein, the expression “dry crystalline form” refers to a polymorph that was dried using any conventional techniques to remove residual solvent. Examples of such conventional techniques can be, but are not limited to, evaporation, vacuum drying, oven drying, drying under nitrogen flow, etc.

The term “solvate”, as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

The amount of solvent employed in a chemical process, e.g., a reaction or a crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding MTBE (1.5 v/v) to a 100 ml reaction mixture would indicate that 150 mL of MTBE was added.

As used herein, the term “reduced pressure” refers to a pressure of about 10 mbar to about 50 mbar.

As used herein, a monohydrate form of Neratinib maleate may be characterized by X-ray diffraction peaks at the following angles (±0.20 degrees) of 2-theta in its X-ray diffraction pattern: 6.53, 8.43, 10.16, 12.19, 12.47, 13.01, 15.17, 16.76, 17.95, 19.86, 21.11, 21.88, 23.22, 23.78, 25.69, 26.17, 27.06, 27.58, 28.26, 28.73, and 29.77.

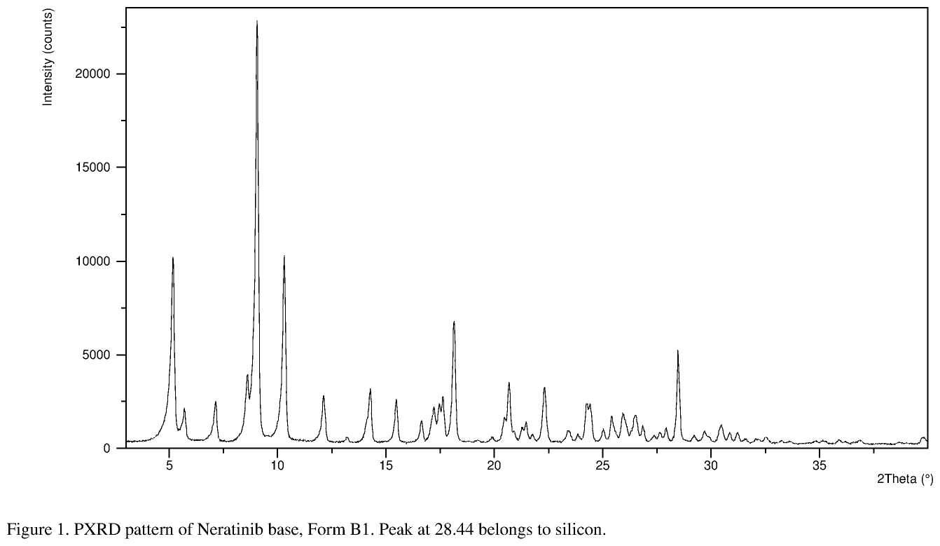

The present disclosure comprises a crystalline form of Neratinib base designated as form B1. The crystalline form B1 of Neratinib base can be characterized by data selected from one or more of the following: a PXRD pattern having peaks at 5.2, 9.1, 10.3, 14.3 and 18.1 degrees 2-theta±0.2 degrees 2-theta; a PXRD pattern as depicted in FIG. 1; or combinations of these data.

Crystalline form B1 of Neratinib base may be further characterized by the PXRD pattern having peaks at 5.2, 9.1, 10.3, 14.3 and 18.1 degrees 2-theta±0.2 degrees 2-theta, and also having one, two, three, four or five additional peaks at 8.6, 15.5, 20.7, 22.3 and 24.2 degrees 2-theta±0.2 degrees 2-theta; or combinations of these data.

Crystalline form B1 of Neratinib base may be characterized by each of the above characteristics alone/or by all possible combinations, e.g., by PXRD pattern having peaks at 5.2, 9.1, 10.3, 14.3 and 18.1 degrees 2-theta±0.2 degrees 2-theta and a PXRD pattern as depicted in FIG. 1.

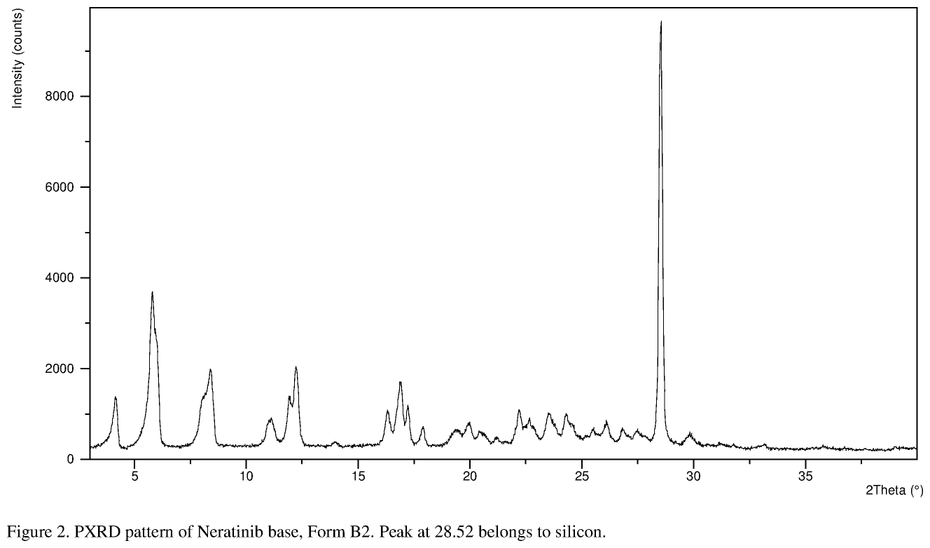

The present disclosure comprises a crystalline form of Neratinib base designated as form B2. The crystalline form B2 of Neratinib base can be characterized by data selected from one or more of the following: a PXRD pattern having peaks at 4.1, 5.7, 8.4, 12.1 and 16.9 degrees 2-theta±0.2 degrees 2-theta; a PXRD pattern as depicted in FIG. 2; or combinations of these data.

Crystalline form B2 of Neratinib base may be further characterized by the PXRD pattern having peaks at 4.1, 5.7, 8.4, 12.1 and 16.9 degrees 2-theta±0.2 degrees 2-theta, and also having one, two, three, four or five additional peaks at 7.9, 11.8, 16.2, 17.1 and 26.0 degrees 2-theta±0.2 degrees 2-theta; or combinations of these data.

Crystalline form B2 of Neratinib base may be characterized by each of the above characteristics alone/or by all possible combinations, e.g., by PXRD pattern having peaks at 4.1, 5.7, 8.4, 12.1 and 16.9 degrees 2-theta±0.2 degrees 2-theta and a PXRD pattern as depicted in FIG. 2.

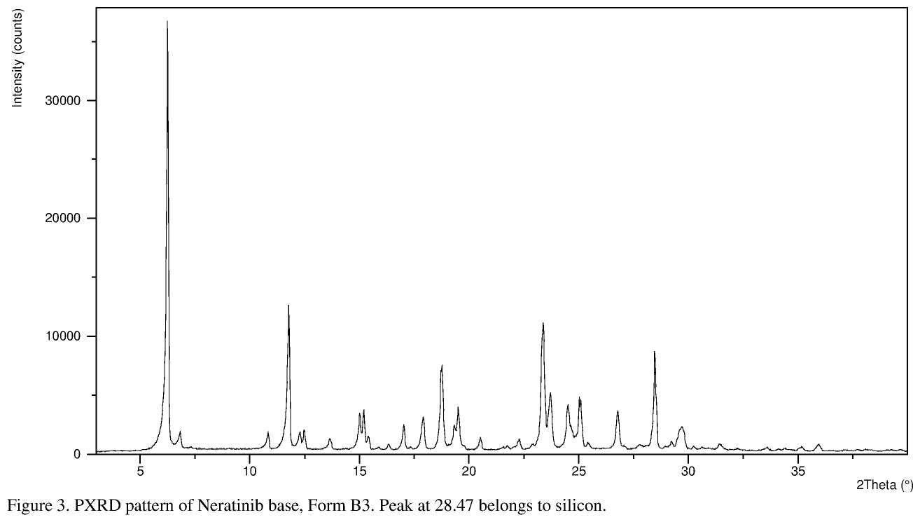

The present disclosure comprises a crystalline form of Neratinib base designated as form B3. The crystalline form B3 of Neratinib base can be characterized by data selected from one or more of the following: a PXRD pattern having peaks at 6.2, 11.7, 18.7, 23.3 and 23.5 degrees 2-theta±0.2 degrees 2-theta; a PXRD pattern as depicted in FIG. 3; or combinations of these data.

Crystalline form B3 of Neratinib base may be further characterized by the PXRD pattern having peaks at 6.2, 11.7, 18.7, 23.3 and 23.5 degrees 2-theta±0.2 degrees 2-theta, and also having one, two, three, four or five additional peaks at 18.5, 19.5, 23.7, 25.0 and 29.5 degrees 2-theta±0.2 degrees 2-theta; or combinations of these data.

Crystalline form B3 of Neratinib base may be characterized by each of the above characteristics alone/or by all possible combinations, e.g., by PXRD pattern having peaks at 6.2, 11.7, 18.7, 23.3 and 23.5 degrees 2-theta±0.2 degrees 2-theta and a PXRD pattern as depicted in FIG. 3.

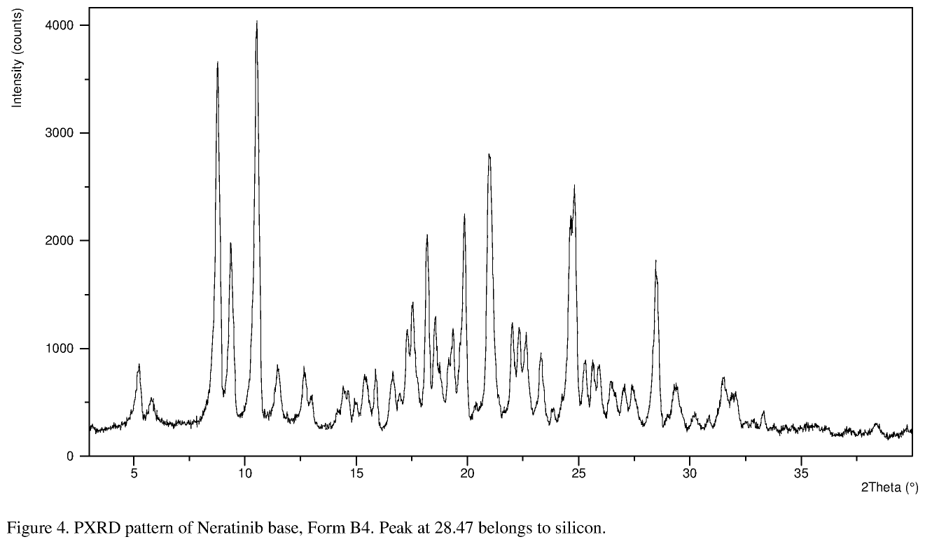

The present disclosure comprises a crystalline form of Neratinib base designated as form B4. The crystalline form B4 of Neratinib base can be characterized by data selected from one or more of the following: a PXRD pattern having peaks at 8.7, 10.5, 19.8, 20.9 and 24.8 degrees 2-theta±0.2 degrees 2-theta; a PXRD pattern as depicted in FIG. 4; or combinations of these data.

Crystalline form B4 of Neratinib base may be further characterized by the PXRD pattern having peaks at 8.7, 10.5, 19.8, 20.9 and 24.8 degrees 2-theta±0.2 degrees 2-theta, and also having one, two, three, four or five additional peaks at 9.3, 17.5, 18.2, 22.0 and 24.6 degrees 2-theta±0.2 degrees 2-theta; or combinations of these data.

Crystalline form B4 of Neratinib base may be characterized by each of the above characteristics alone/or by all possible combinations, e.g., by PXRD pattern having peaks at 8.7, 10.5, 19.8, 20.9 and 24.8 degrees 2-theta±0.2 degrees 2-theta and a PXRD pattern as depicted in FIG. 4.

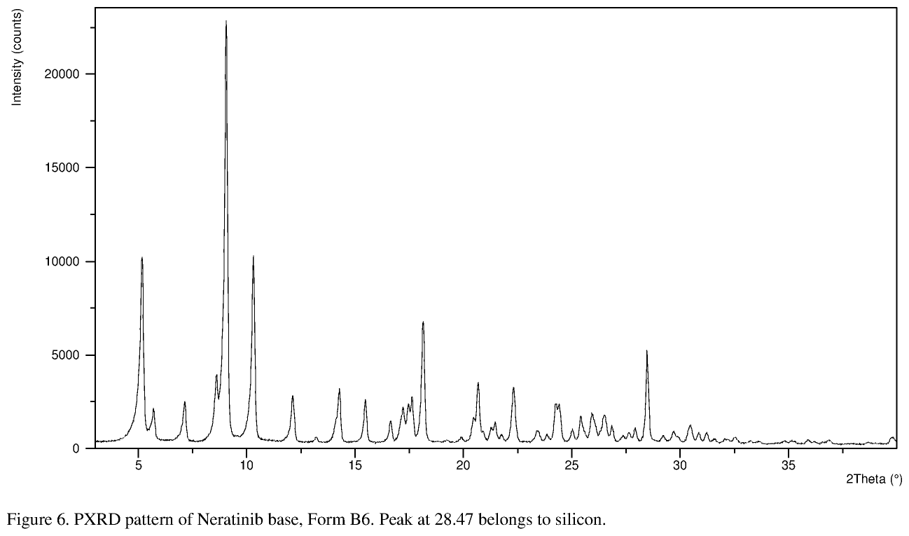

The present disclosure comprises a crystalline form of Neratinib base designated as form B6. The crystalline form B6 of Neratinib base can be characterized by data selected from one or more of the following: a PXRD pattern having peaks at 6.2, 11.6, 23.0, 23.8 and 24.7 degrees 2-theta±0.2 degrees 2-theta; a PXRD pattern as depicted in FIG. 6; or combinations of these data.

Crystalline form B6 of Neratinib base may be further characterized by the PXRD pattern having peaks at 6.2, 11.6, 23.0, 23.8 and 24.7 degrees 2-theta±0.2 degrees 2-theta, and also having one, two, three, four or five additional peaks at 6.8, 7.2, 10.7, 12.3 and 15.6 degrees 2-theta±0.2 degrees 2-theta; or combinations of these data.

Crystalline form B6 of Neratinib base may be further characterized by the PXRD peak list as provided in Table 1.

TABLE 1
XRPD peaks (degrees two theta)

6.1
6.8
7.2
10.7
11.6
12.3
13.6
15.0
15.2
15.6
16.1
16.9
17.1
17.7
18.5
19.0
19.4
19.5
20.4
21.3
21.6
22.0
22.6
23.0
23.3
23.8
24.7
25.3
26.8
27.2
27.5
28.1
28.5
28.8
29.1
29.4
29.9
30.2

Crystalline form B6 of Neratinib base may be characterized by each of the above characteristics alone/or by all possible combinations, e.g., by PXRD pattern having peaks at 6.2, 11.6, 23.0, 23.8 and 24.7 degrees 2-theta±0.2 degrees 2-theta and a PXRD pattern as depicted in FIG. 6.

Crystalline form B6 of Neratinib base may be polymorphically pure or substantially free of any other solid state (or polymorphic) forms.

Crystalline form B6 of Neratinib base shows similar solubility to Neratinib maleate monohydrate. Solubility is an important molecular property that influences the intestinal absorption, and is necessary for optimization of formulation The present invention describes crystalline form B6 of Neratinib base having solubility properties in a good correlation with Neratinib maleate monohydrate, therefor may be preferred alternative to a salt formation.

Furthermore, the density of crystalline form B6 of Neratinib base is higher than that of the Neratinib maleate monohydrate. In correlation, the compressed density index (CDI) of crystalline form B6 of Neratinib base is lower than of Neratinib maleate monohydrate. CDI value can be used to index the ability of powder to flow (Arne Hagsten Sørensen, Jørn Møller Sonnergaard & Lars Hovgaard (2005); Bulk Characterization of Pharmaceutical Powders by Low-Pressure Compression, Pharmaceutical Development and Technology, 10:2, 197-209). CDI value of crystalline form B6 of Neratinib base is below 15 wherein CDI of less than 15 is considered a sign of good flowability. Accordingly crystalline form B6 of Neratinib base having higher density is typically less cohesive and therefore exhibits better filterability and flowability.

Good filterability is a prerequisite for enabling the production of the API on an industrial scale. A good flowability of a powder is particularly important in the high-volume formulation of the API into solid dosage forms, which necessitates rapid, uniform and consistent filling of cavities such as capsules, or tablet presses. Poor flow characteristics cause slow and nonuniform press feeding and difficulties in ensuring a consistent, reproducible fill of the cavities.

Therefore, the crystalline form B6 of Neratinib base of the present invention has favorable technological (physical and mechanical) properties, which offers advantages during handling and processing, e.g., in tablet formulation processes.

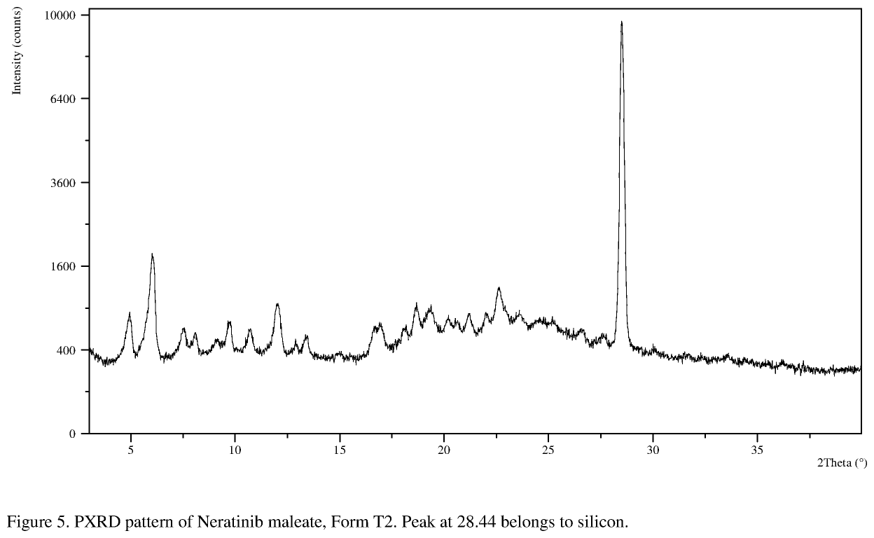

The present disclosure comprises a crystalline form of Neratinib maleate designated as form T2. The crystalline form T2 of Neratinib maleate can be characterized by data selected from one or more of the following: a PXRD pattern having peaks at 4.9, 6.1, 12.1, 18.7 and 22.7 degrees 2-theta±0.2 degrees 2-theta; a PXRD pattern as depicted in FIG. 5; or combinations of these data.

Crystalline form T2 of Neratinib maleate may be further characterized by the PXRD pattern having peaks at 4.9, 6.1, 12.1, 18.7 and 22.7 degrees 2-theta±0.2 degrees 2-theta, and also having one, two, three, four or five additional peaks at 19.4, 20.2, 21.2, 22.0 and 23.6 degrees 2-theta±0.2 degrees 2-theta; or combinations of these data.

Crystalline form T2 of Neratinib maleate may be characterized by each of the above characteristics alone/or by all possible combinations, e.g., by PXRD pattern having peaks at 4.9, 6.1, 12.1, 18.7 and 22.7 degrees 2-theta±0.2 degrees 2-theta and a PXRD pattern as depicted in FIG. 5.

The present disclosure also provides the use of the solid state forms of Neratinib base and Neratinib salts, preferably maleate salt, for preparing other solid state forms of Neratinib, Neratinib salts and solid state forms thereof. In particular, the present disclosure further comprises use of crystalline form B6 of Neratinib base according to any proceeding embodiment for preparing crystalline form of Neratinib maleate or other pharmaceutically acceptable salt, for example Neratinib maleate monohydrate or anhydrous Neratinib maleate.

The present disclosure further encompasses processes for preparing Neratinib salts or solid state forms thereof. The process comprises preparing any of the solid state forms of Neratinib base of the present disclosure, and converting it to Neratinib salt. The conversion can be done, for example, by a process comprising reacting the obtained Neratinib base with an appropriate acid to obtain the corresponding acid addition salt.

In another embodiment the present disclosure encompasses the above described solid state forms of Neratinib base, Neratinib salts, preferably maleate salt or combinations thereof, for use in the preparation of pharmaceutical compositions and/or formulations, preferably for the treatment of HER2-positive solid tumors, including breast cancer and non-small cell lung cancer (NSCLC).

In another embodiment the present disclosure encompasses the use of the above described solid state forms of Neratinib base, Neratinib salts, preferably maleate salt or combinations thereof, for the preparation of pharmaceutical compositions and/or formulations.

The present disclosure further provides pharmaceutical compositions comprising the solid state forms of Neratinib base, Neratinib salts, preferably maleate salt or combinations thereof, according to the present disclosure.

In yet another embodiment, the present disclosure encompasses pharmaceutical formulations comprising at least one of the above described solid state forms of Neratinib base or Neratinib salts, preferably maleate salt, and at least one pharmaceutically acceptable excipient.

The present disclosure encompasses a process to prepare said formulations of Neratinib comprising combining at least one of the above solid state forms and at least one pharmaceutically acceptable excipient.

The solid state forms as defined herein, as well as the pharmaceutical compositions or formulations of Neratinib can be used as medicaments, particularly for the treatment of HER2-positive solid tumors, including breast cancer and non-small cell lung cancer (NSCLC).

The present disclosure also provides a method of treating of HER2-positive solid tumors, including breast cancer and non-small cell lung cancer (NSCLC), comprising administering a therapeutically effective amount of the solid state form of Neratinib base or Neratinib salts, preferably maleate salt, of the present disclosure, or at least one of the above pharmaceutical compositions or formulations, to a subject suffering from HER2-positive solid tumors, including breast cancer and non-small cell lung cancer (NSCLC), or otherwise in need of the treatment.

The present disclosure also provides the use of the solid state forms of Neratinib base or Neratinib salts, preferably maleate salt, of the present disclosure, or at least one of the above pharmaceutical compositions or formulations for the manufacture of a medicament for treating HER2-positive solid tumors, including breast cancer and non-small cell lung cancer (NSCLC).

Having described the invention with reference to certain preferred embodiments, other embodiments will become apparent to one skilled in the art from consideration of the specification. The invention is further illustrated by reference to the following examples describing in detail the preparation of the composition and methods of use of the invention. It will be apparent to those skilled in the art that many modifications, both to materials and methods, may be practiced without departing from the scope of the invention.

### Analytical Methods

### Powder X-Ray Diffraction Pattern (“PXRD”) Method:

Sample after being powdered in a mortar and pestle is applied directly on a silicon plate holder. The X-ray powder diffraction pattern was measured with Philips X'Pert PRO X-ray powder diffractometer, equipped with Cu irradiation source=1.54184 Å (Ångström), X'Celerator (2.022° 2θ) detector. Scanning parameters: angle range: 3-40 deg., step size 0.0167, time per step 37 s, continuous scan. The described peak positions were determined using silicon powder as an internal standard in an admixture with the sample measured. The position of the silicon (Si) peak was corrected to silicone theoretical peak: 28.45 degrees two theta and the positions of the measured peaks were corrected respectively.

### EXAMPLES

The starting material Neratinib maleate may be obtained according to the process described in U.S. Pat. No. 8,022,216.

### Example 1

### Preparation of Neratinib Base Form B3

Neratinib maleate monohydrate (20.0 g) was suspended in 1000 mL of 96% ethanol. Suspension was heated up to 73° C. and into the obtained solution 110 mL of 10% v/v solution of triethylamine in acetonitrile (ACN) was added dropwise. Afterwards, 1000 mL of water was added to the solution dropwise. Solution was cooled to RT and stirred for additional 4 hours. Obtained suspension was filtered and wet product was dried at 60° C. and 50 mbar for 4 hours. Neratinib base Form B3 was confirmed by PXRD as presented in FIG. 3.

### Example 2

### Preparation of Neratinib Base Form B1

Neratinib base form B3 (50 mg) was suspended in methanol (1 mL) and heated at 60° C. for 1 hour. Suspension was cooled to room temperature and filtered. Neratinib base form B1 has been confirmed by PXRD as presented in FIG. 1.

### Example 3

### Preparation of Neratinib Base Form B1

Neratinib base form B3 (2.5 g) was suspended in 50 mL of methanol, heated to 60° C. for 1 hour and left to stand at RT for 3 hours. Suspension was filtered and Neratinib base form B1 has been confirmed by PXRD.

### Example 4

### Preparation of Neratinib Base Form B2

Neratinib base form B3 (50 mg) was suspended in tetrahydrofuran (THF, 1 mL) and heated at 60° C. for 1 hour. Suspension was cooled to room temperature and filtered. Neratinib base form B2 has been confirmed by PXRD as presented in FIG. 2.

### Example 5

### Preparation of Neratinib Base Form B2

Neratinib base form B3 (2.5 g) was suspended in 50 mL of tetrahydrofuran, heated to 60° C. for 1 hour and left to stand at RT for 3 hours. Suspension was filtered and Neratinib base form B2 has been confirmed by PXRD.

### Example 6

### Preparation of Neratinib Base Form B4

Neratinib base form B3 (50 mg) was suspended in ethyl acetate (1 mL) and heated at 80° C. for 1 hour. Suspension was cooled to room temperature and filtered. Neratinib base form B4 has been confirmed by PXRD as presented in FIG. 4.

### Example 7

### Preparation of Neratinib Base Form B4

Neratinib base form B3 (2.5 g) was suspended in 50 mL of isobutyl acetate, heated to 100° C. for 1 hour and left to stand at RT for 3 hours. Suspension was filtered and Neratinib base form B4 has been confirmed by PXRD.

### Example 8

### Preparation of Neratinib Maleate Form T2

5 g of Neratinib maleate (monohydrate) was suspended in 100 mL of cyclohexanone and heated to 120° C. until dissolved. Solution was cooled to RT. Suspension formed after cooling was filtered and crude product suspended in heptane at RT and stirred for 30 min. Washed crystals were filtered and suspended in cyclopentane for 30 min, and isolated by filtration. Wet product was dried in vacuum oven at 50° C. and 50 mbar for 4 hours. Neratinib maleate form T2has been confirmed by PXRD as presented in FIG. 5.

### Example 9

### Preparation of Neratinib Base Form B6

Neratinib base form B1 (30 g) was suspended in mixture of tetrahydrofuran (228 mL) and water (12 mL). Suspension was heated to reflux (65-70° C.). Obtained solution was cooled to 50 -55° C. and stirred for 3 h—crystallization occurs. Suspension was cooled to 0 -5° C. during 2 h and stirred for 2 h at that temperature. Crystals were filtered and washed with 2×30 mL of water and dried under vacuum (10 mbar) at 50° C. 16 h. Neratinib base form B6 (Yield: 88% %; Purity: 99.8 A %) has been confirmed by PXRD as presented in FIG. 6.

### Example 10

### Preparation of Neratinib Base Form B6

Neratinib base (10 g) was suspended in mixture of acetonitrile (90 mL), tetrahydrofuran (60 mL) and water (20 mL). Suspension was heated to reflux (70-75° C.). Obtained solution was cooled to 50-55° C. and stirred for 3 h—crystallization occurs. Suspension was cooled to 0-5° C. during 1 h and stirred for 2 h at that temperature. Crystals were filtered and washed with 2×15 mL of acetonitrile/tetrahydrofuran (1.5:1) and dried under vacuum (10 mbar) at 50° C. 16 h. Neratinib base form B6 (Yield: 85%; Purity: 99.8 A %) has been confirmed by PXRD.

### Example 11

### Preparation of Neratinib Base Form B1

(E)-4-(dimethylamino)but-2-enoic acid hydrochloride (8.92 g; 53.82 mmoL) was charged to glass reactor (250 mL) inertized with nitrogen. Dichloromethane (85 mL) and dimethylformamide (DMF; 318 μL; 4.12 mmoL) were added. Suspension was cooled to 0-5° C. and oxalyl chloride (4.0 mL; 45.76 mL) was added dropwise during 5-10 min. Dropping funnel was washed with dichloromethane (5 mL). Reaction mixture was stirred for 8-9 h in nitrogen atmosphere and monitored by HPLC. After finish of reaction, solution of 6-amino-4-((3-chloro-4-(pyridin-2-ylmethoxy)phenyl)amino)-7-ethoxyquinoline-3-carbonitrile (12.0 g; 26.91 mmoL) in N-methyl-2-pyrrolidone (NMP, 120 mL) was added dropwise during 15-30 min while maintaining the temperature below 10° C. Reaction mixture was heated to 20-25° C. and stirred for 2-16 h (reaction was monitored by HPLC until NRT-3 was below 0.2 Area %). After completion of reaction, water (120 mL) was added and layers separated. Into water layer NMP (48 mL) and THF (96 mL) were added and pH was adjusted to 10.0-10.5 by addition of 2 M NaOH while maintaining the temperature at 20-25° C. Mixture was stirred at 20-25° C. for 2 h, cooled to 0-5° C. during 1 h, stirred for 2 h and filtered. Crystals were washed with THF/water mixture (1:2; 2×15 mL) and dried under vacuum at 50° C. for 16 at 10 mbar to obtain Neratinib base form B1 (Y:95%).

### Example 12

### Preparation of Neratinib Base Form B6

Neratinib base form B1 (13.5 g) was dissolved in ACN/THF/water mixture (9:6:2; 220 mL) while heating to 70-75° C. Solution was cooled to 50-55° C. during 15 min, stirred at 50-55° C. for 3 h (crystallization occurs) and cooled to 0-5° C. during 1 h. Suspension was stirred at 0-5° C. for 2 h and filtered. Crystals were washed with ACN/water mixture (1:2; 2×20 mL) and dried under vacuum at 50° C. for 16 h at 10 mbar to obtain Neratinib base form B6 (Y: 92.4%; HPLC purity: 99.71 Area %).

### Example 13

### Preparation of Neratinib Base Form B6

Neratinib base form B1 (4 g) was dissolved in THF/water mixture (19:1; 31.6 mL) while heating to 65-70° C. Solution was cooled to 50-55° C. during 15 min, stirred at 50-55° C. for 4 h (crystallization occurs) and cooled to 0-5° C. during 1 h. Suspension was stirred at 0-5° C. for 2 h and filtered. Crystals were washed with water (2×5 mL) and dried under vacuum at 50° C. for 16 at 10 mbar to obtain Neratinib base form B6 (Y: 92.3%; HPLC purity: 99.84 Area %).

### Example 14

### Preparation of Neratinib Maleate Monohydrate

Neratinib base form B6 (11.5 g) and maleic acid (2.39 g) were charged and methanol/water mixture (4:1; 47.5 mL) was added. Mixture was stirred at 20-25° C. for 3 h. Water (34.5 mL) was added dropwise and suspension was stirred for additional 1 h. Suspension was filtered and crystals were washed with methanol/water mixture (1:2; 2×15 mL). Crystals were dried under vacuum at 35° C. for 6 h at 10 mbar to obtain Neratinib maleate monohydrate (Y: 84.6%; HPLC purity: 99.76 Area %).

### Example 15

### Preparation of Neratinib Maleate Monohydrate

Neratinib base form B6 (11.5 g) and maleic acid (2.39 g) were charged and methanol/water mixture (20:1; 92 mL) was added. Mixture was stirred at 20-25° C. for 3 h. Suspension was filtered and crystals were washed with methanol/water mixture (1:1; 15 mL) and with water (2×20 mL). Crystals were dried under vacuum at 35° C. for 6 h at 10 mbar to obtain Neratinib maleate monohydrate (Y: 78.6%; HPLC purity: 99.75 Area %).

### Example 16

### Preparation of Neratinib Maleate Monohydrate

Neratinib base form B6 (11.5 g; 20.64 mmol) and maleic acid (2.39 g; 20.59 mmol) were charged in three-necked flask (250 mL) equipped with mechanical stirrer. Methanol/water mixture (20:1; 92 mL) was added and mixture was stirred at 20-25° C. for 3 h. Suspension was filtered, crystals were washed with methanol/water mixture (1:1; 15 mL) and then suspended in water (50 mL) and filtered again. Crystals were dried under vacuum at 35° C. for 6 h at 10 mbar to obtain Neratinib maleate monohydrate (Y: 79.8% HPLC purity: 99.88 Area %).

### Example 17

### Preparation of Neratinib Maleate Anhydrous

Neratinib base form B6 (10 g) and maleic acid (2.15 g) were charged and methanol/water mixture (20:1; 84 mL) was added. Mixture was stirred at 20-25° C. for 4 h and cooled to 0-5° C. during 2 h. Suspension was stirred for 10 h at 0-5° C. and filtered. Crystals were washed with cold methanol (2×10 mL) to obtain Neratinib maleate anhydrous.

### Example 18

### Preparation of Neratinib Maleate Anhydrous

Neratinib base form B6 (5 g) and maleic acid (1.07 g) were charged and methanol/water mixture (30:1; 39.25 mL) was added. Mixture was stirred at 20-25° C. for 4 h and cooled to 0-5° C. Suspension was stirred for 2 h at 0-5° C. and filtered. Crystals were washed with cold methanol (2×5 mL) to obtain Neratinib maleate anhydrous.

### Example 19

### Solubility Test

Solubility test was performed according to European Pharmacopoeia 7.0, page 637, 5.11.

Comparison of solubility between Neratinib base B6 (prepared according to example 12) and Neratinib maleate monohydrate is provided in the table below.

| | NRT maleate monohydrate | Neratinib base B6 |
|---|---|---|
| pH = 1.1 | sparingly | sparingly |
| pH = 4.5 | slightly | slightly |
| pH = 6.8 | very slightly | very slightly |

Solubility shows good correlation of Neratinib base form B6 and Neratinib maleate monohydrate under all tested conditions.

### Example 20

### Texture Analysis

A simple and low sample consuming method for evaluation of the packing density of powders were obtained as compression profiles under low pressures using a die and a flat-faced punch fitted on a TA-XTplus Texture analyser (Stable Micro Systems Ltd., Godalming, UK). The small amount of 200 mg of sample was compressed in a steel mould (with the rate of displacement 0.03 mm/s). Also, a cyclic procedure (similar to tapping) was performed at maximal compressive displacement 0.5 mm, then retracting, relaxation for 15 s and then repeated compressive steps (altogether up to 20 steps).

Density at 0.2 MPa and CDI data of Neratinib maleate Form monohydrate and Neratinib maleate Form B6 (prepared according to example 12) are presented in Table 2.

TABLE 2
Results of Texture analyzer test

| | Density at | Compressed density |
|---|---|---|
| Compound name | 0.2 MPa/g/m³ | index, CDI |
| Neratinib maleate monohydrate | 0.4452 | 14.55 |
| Neratinib base Form B6 | 0.5577 | 10.89 |

1. Crystalline form B6 of Neratinib base, characterized by data selected from one or more of the following: a PXRD pattern having peaks at 6.2, 11.6, 23.0, 23.8 and 24.7 degrees 2-theta±0.2 degrees 2-theta; a PXRD pattern as depicted in FIG. 6; or combinations of these data.

2. Crystalline form B6 of Neratinib base according to any claim 1, further characterized by the PXRD pattern having one, two, three, four or five additional peaks at 6.8, 7.2, 10.7, 12.3 and 15.6 degrees 2-theta±0.2 degrees 2-theta.

3. Crystalline form B6 of Neratinib base according to claim 1, which is polymorphically pure or substantially free of any other solid state forms of Neratinib base or salts thereof.

4. Crystalline form B6 of Neratinib base according to claim 1 containing: 20% (w/w) or less of any other solid state forms of Neratinib base or salts thereof.

5. (canceled)

6. A process for the preparation of Neratinib maleate or solid forms thereof, wherein the process comprises preparing the crystalline for B6 of Neratinib base defined in claim 1, and converting it to Neratinib maleate.

7. (canceled)

8. Process for the preparation of Neratinib salts or solid state forms thereof, wherein the process comprises preparing the crystalline form B6 of Neratinib base defined in claim 1, and converting it to Neratinib salt.

9-10. (canceled)

11. A pharmaceutical compositions comprising crystalline form B6 of Neratinib base defined in claim 1.

12. A pharmaceutical formulations comprising crystalline form B6 of Neratinib base defined in claim 1, and at least one pharmaceutically acceptable excipient.

13. A process to prepare said formulations of Neratinib comprising combining crystalline form B6 of Neratinib base defined in claim 1, optionally converting it to Neratinib salt, and at least one pharmaceutically acceptable excipient.

14-15. (canceled)

16. A method of treating of HER2-positive solid tumors comprising administering a therapeutically effective amount of crystalline form B6 of Neratinib base defined in claim 1, or at least one of the above pharmaceutical compositions or formulations defined in claims 11-12, to a subject suffering from HER2-positive solid tumors, or otherwise in need of the treatment.
